(19) United States
(12) Patent Application Publication
(10) Pub. No.: US 2017/0231529 A1
(43) Pub. Date: Aug. 17, 2017

(54) METHOD AND APPARATUS FOR ASSISTING MOVEMENT REHABILITATION

(71) Applicant: National University of Singapore, Singapore (SG)

(72) Inventors: Shi-Cheng Yen, Singapore (SG); Arthur Ee Beng Tay, (US); Choon Huat Gerald Koh, Singapore (SG)

(73) Assignee: National University of Singapore, Singapore (SG)

(21) Appl. No.: 15/504,651

(22) PCT Filed: Aug. 18, 2015
(86) PCT No.: PCT/SG2015/000136

§ 371 Date: Feb. 16, 2017

Related U.S. Application Data
(60) Provisional application No. 62/038,532, filed on Aug. 18, 2014.

(51) Int. Cl. A61B 5/11; A61B 5/00

(52) U.S. Cl. CPC A61B 5/1114; A61B 5/0022; A61B 5/746; A61B 5/486

(57) ABSTRACT

A method and apparatus implementing a tele-rehabilitation system with remote supervision via wireless telecommunication means is disclosed. The method for assisting a user performing musculoskeletal movement in rehabilitation exercise comprises of motion-sensing a first part of user's body to be exercised (e.g. upper or lower limb), and motion-sensing a second part of user's body not intended to be moved (e.g. trunk or neck) during the first part of user's body being exercised. The method includes alerting the user of undesirable movements (i.e. compensation) sensed in the trunk or neck in real-time and with biofeedback to the user. The sensors transmit data wirelessly (e.g. via Bluetooth) to be processed and/or stored by a computing device nearby such as a tablet, smart-phone or laptop, and processes the data for efficient visual representation of the rehabilitation exercise. The method also captures video recordings of the user performing the exercises for review by the supervising therapist. It may transmit the data over the Internet to a rehabilitation supervision centre where a supervising therapist will be able to use the visual representations of the exercises to monitor the exercises of the user efficiently (including tracking compensation), and reviewing the video recordings of the exercises where necessary. The apparatus also includes video conferencing capability for the therapist to link with the user for real-time rehabilitation guidance. Simultaneous data transmission sessions of multiple users may be conducted with a therapist.

### TECHNICAL FIELD

The present invention relates to a method for assisting movement rehabilitation of users suffering from motor disability, including post-stroke patients. The method alerts users to unintended motion such as compensation movements during rehabilitation exercises. Apparatus configured to implement the method is also disclosed.

### BACKGROUND ART

The incidence of stroke in Singapore is about 10,000 strokes a year and the prevalence of stroke in the general population is about 7.7% which is expected to continue to increase significantly due to a rapidly ageing population. In co-inventor Dr Gerald Koh's one-year cohort study on local post-stroke patients, patients that underwent follow-up supervised therapy recovered faster and achieved a higher functional status at the end of one year.

However, only 25% of the patients were able to undergo such supervised rehabilitation one month after their discharge. The reasons for inability to undergo such rehabilitation include physical, social and financial factors. In addition, home visits by therapists are extremely time consuming and expensive, i.e. at about 2-3 patients/day at home compare to 15-20 patients/day at the hospital. If these burdens can be lessened so that patients are able to continue rehabilitation, they can often significantly reduce their disability and even make a full recovery. The resulting cost savings to Singapore will be highly significant.

Besides, there is also a shortage of rehabilitation therapists which results in crowded rehabilitation wards and long wait times for patients. These are also factors that discourage patients from continuing with their rehabilitation. As many of these barriers are difficult to overcome, there is a long-felt need for home-based automated supervised rehabilitation systems using the wide-availability of telecommunication technologies so that patients do not have to travel to the rehabilitation centre or clinic.

The need for supervision in the rehabilitation of a stroke patient is to ensure that the exercises performed are correct in order to obtain the maximum benefit. Very often, patients do not perform their prescribed exercises correctly. For instance, patients often compensate for their inability to lift their arms by arching the rest of the body. This compensation, if not corrected promptly, can develop into other problems such as poor coordination, gait and even injuries. The situation is complicated if these patients only meet up with their therapist once a week, hence the wrong movement would not be promptly corrected and would become habitual or reinforced. There exist tele-rehabilitation systems that use video monitoring exclusively which require the therapist to watch the patient in real-time, or to review hours of footage to spot problems such as compensation. These systems provide little improvement over face-to-face or one-to-one supervision at the clinic or rehabilitation centre.

### SUMMARY OF INVENTION

There is thus a need for a remote supervisory means for a patient conducting by himself rehabilitation exercises such as musculoskeletal movement, e.g. of the limbs, whereby any undesirable or unintentional compensation made by the patient could be noticed and corrected by the supervising therapist. It is also desirable that such a rehabilitation system does not require the user to don too many sensor patches with wires attached that might be distracting and encumbering movement.

We have now developed a tele-rehabilitation system with supervision via wireless telecommunication means as defined hereinafter in the patents claims which are directed to the method and apparatus implementing the system.

In the first aspect of our invention, the method for assisting a user performing musculoskeletal movement in rehabilitation exercise generally comprises of motion-sensing a first part of user's body to be exercised, motion-sensing a second part of user's body not intended to be moved during the first part of user's body being exercised, and alerting the user of undesirable compensatory movements sensed of said second part of user's body.

In one preferred embodiment of the method, the motion-sensing of at least one of the first part and second part of user's body is transduced into wireless signal and transmitted for data processing and data storage. The alerting of undesirable compensatory movements may be made in the form of real-time biofeedback to the user, including any one or combination of visual, audio or vibration, said biofeedback of intensity sufficient to be perceivable by the user as a stroke patient. Preferably, the alerting of unintended movement to the user is made as part of the data processing.

In another preferred embodiment, the first part of user's body is a limb to be exercised and the second part is a point on user's body most likely subject to undesirable compensatory movements. The motion-sensing of the first part of user's body may preferably be sensed at a volitional torque measurable point of the limb to be exercised, and the motion-sensing of the second part of the user's body against undesirable compensatory movements is sensed at the user's neck. Preferably, the step of detecting undesirable compensatory movements is made at the user's neck in relation to the limb's movements, and the step of transducing both movements into signals and transmitting said signals are processed into visual representation of the rehabilitation exercise session, wherein the visual representation comprises plotting each of the movements in series and align both movement series by time-frame.

The data processing aspect of our invention may include any one or combination of alerting of undesirable compensatory movements by the user, storing and plotting the data for visual representation of the rehabilitation exercise, transmitting aforesaid data to a rehabilitation supervision centre, and optionally receiving data from said rehabilitation supervision centre.

Another preferred embodiment of the data processing may include video conferencing between the user performing the rehabilitation exercise and the rehabilitation supervision centre to enable a therapist to view the user's exercises and providing instructions or guidance to him in real-time. Preferably, the data transmission is conducted via web-based server processing accessible by the user and therapist. The therapist may also be advantageously placed in multiple simultaneous data transceiving (i.e. transmitting and receiving) sessions with a plurality of such connected users.

In a second aspect of our invention, the apparatus for assisting a user to perform musculoskeletal movement in rehabilitation exercise has the general embodiment comprising a first motion sensor attachable to a first part of user's body to be exercised, a second motion sensor attachable to a second part of user's body not intended to be moved during the first part of user's body being exercised, and an alarm alerting the user of undesirable compensatory movements sensed of the second part of user's body in relation to the first part of the user's body being exercised. Preferably, at least one of the first and second motion sensors may transduce motion sensed into wireless signal and transmit it to a computing device for data processing and storage.

In one aspect of the apparatus, the alarm may preferably alert the user of making undesirable compensatory movements by way of real-time biofeedback to said user, including any one or combination of visual, audio or vibration, said biofeedback of intensity sufficient to be perceivable by the user as a stroke patient. Preferably, the alarm is triggered as a result of the computing device having processed the data received from the motion sensor indicating undesirable compensatory movements being sensed.

In one preferred embodiment of the apparatus, the first part of the user's body attachable with the first motion sensor is a limb to be exercised and the second part of the user's body attachable with the second motion sensor is a point on the user's body that is most likely subject to undesirable compensatory movements. Preferably still, the motion sensor attachable on the first part of user's body is attached to a volitional torque-measurable point of the limb to be exercised, and the motion sensor attachable to the second part of the user's body for detecting undesirable compensatory movements is attached to the user's neck.

Another aspect of the apparatus concerns the computing device which provides data processing for any one or combination of alarm triggered upon undesirable compensatory movements by the user, data storage and plotting to generate visual representation of the rehabilitation exercise, transmitting aforesaid data to a rehabilitation supervision centre; and optionally receiving data from said rehabilitation supervision centre.

In one preferred embodiment of the apparatus, the motion sensor detecting undesirable compensatory movements is adapted to be donned at the user's nape with the detection in relation to the limb's movement, and the computing device receiving signals of both movements is operable to process said signals into visual representation of the rehabilitation exercise session, wherein the visual representation comprises charts of each of the movements in series and both movement series' charts are aligned by time-frame. Preferably, the computing device includes video camera which allows video conferencing between the user performing the rehabilitation exercise and the rehabilitation supervision centre, enabling a therapist to view the user's exercises and providing instructions or guidance to the user in real-time.

In yet another aspect of the apparatus, the data transmission between the computing device at user's end and the rehabilitation supervision centre is conducted via a web-based server which is accessible by said user and therapist. Preferably, simultaneous data transceiving sessions of multiple users are conducted with a therapist at the rehabilitation supervision centre.

### BRIEF DESCRIPTION OF DRAWINGS

The drawings accompanying this specification as listed below may provide a better understanding of our invention and its advantages when referred in conjunction with the detailed description the follows as exemplary and non-limiting embodiments of our invention, which is an automated rehabilitation system with supervision via telecommunication means.

FIG. 1 shows a schematic representation of the method and apparatus configuration of our invention.

FIG. 2 is a photograph of actual devices used in our prototype apparatus for implementing our invention.

FIGS. 3(a) and 3(b) illustrate the locations on parts of the user's body for placement of motion sensor according to our invention.

FIG. 4 is a sample list of exercises for upper and lower limbs.

FIGS. 5(a), 5(b) and 5(c) illustrate a trial subject's raising an upper limb for exercise and its compensation.

FIG. 6 displays data plotted of a series of exercises as illustrated in FIGS. 5 above.

FIGS. 7(a) and 7(b) illustrates another trial subject's limb rotation exercises and its compensation.

FIG. 8 displays data plotted of a series of exercises as illustrated in FIGS. 7 above.

### DETAILED DESCRIPTION OF SPECIFIC EMBODIMENTS

Configuration

As shown in FIG. 1 as an overview of a working prototype of our invention, i.e. an automated rehabilitation system with supervision via telecommunication means. The system comprises of a tablet and, preferably, not more than two wireless wearable sensors at the user's end. The sensors comprises of (i) a wearable sensor node to be wore on the limb of a user for monitoring its range-of-motion and provides real-time biofeedback to, the user, and (ii) a compensation [sensor] node, which is recommended to be worn on the back of the neck for detecting the most common motor compensation during exercise.

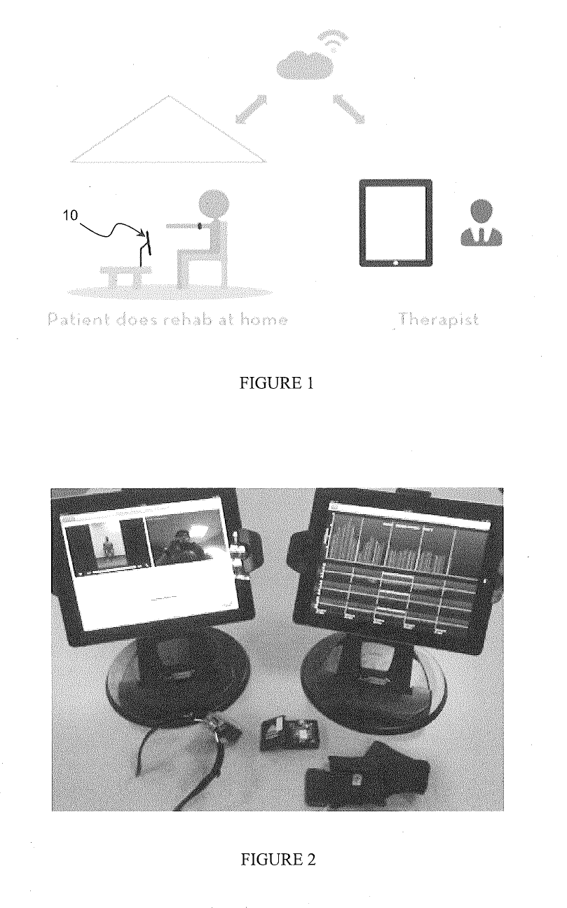

A basic configuration of the apparatus that is capable of implementing our method or system comprises of 2 tablet computers and 2 wearable sensors as shown in FIG. 2. In the photograph of FIG. 2, both tablet computers are iPads—one each for the patient and therapist. It should be noted that the therapist can use the same iPad to remotely supervise multiple patients or users as our system may be scalable upwards by having multiple channels of simultaneous data and video communication so that a plurality of patients may be supervised individually.

It should also be noted that instead of iPads or iOS-based devices, it is also possible to configure our system to run on Android tablets or smartphones or Windows tablets, laptops or PCs as long as the devices meet both short-range communication (e.g. Bluetooth or infrared) as well as long range data communication capability (e.g. 4G or LTE, or has WiFi enabling it to access the internet via a gateway so that it may communicate with the supervising therapist's device setup.

In short, the system is able to monitor if the patient conforms to the exercise regime and if any compensation is made during exercise as detected via the compensation sensor node mounted at the back of the neck of the user. The system also provide biofeedback to advise if the patient has met the target angle in various strengthening exercise. The system also captures video recordings of the users performing the exercises so they can be reviewed by the supervising therapist as needed. Finally, the data may be uploaded to a cloud storage for the therapist to review and adjust the exercise difficulty level.

Sensors

For the example of one-to-one supervision, the 2 wearable sensors are provided for the user. Each of the wearable prototype sensor device is actually a “compound sensor”, i.e. comprising a core set of different sensors—accelerometer, gyroscope and compass—along with the accompanying electronic components controlled by a processor or microcontroller (which in our prototype is an Arduino™ microcontroller available from www.arduino.cc/en/Main/arduinoBoardUno that utilises Bluetooth wireless technology to transmit data wirelessly to a tablet.

The data from both sensor nodes are preferably transmitted wirelessly (and not necessarily via Bluetooth but) via any suitable short-range, low energy radio wave to the tablet which may then process the data and/or uploaded it to a web server. In addition, video recordings of the user performing the exercises may also uploaded to the web server. From there, the data may be presented in a succint summary plot to the therapist to review. If necessary, the therapist may also review the videos of the user performing the exercises. The therapist can then remotely alert and guide the patient using teleconference software on the tablet on how to reduce motor compensation and to progress the user to a new, more challenging set of exercises.

The sensor node, that we have built as a prototype and used in this system is a 9 Degrees of Freedom (DOF) sensor that is mounted on a custom-designed PCB board. The main components of the PCB include: a BMA180 accelerometer, a ITG3200 gyroscope, HMC5843 Magnetometer and an ATMEGA328 microcontroller. The microcontroller samples the sensors for data and is transmitted by a Bluetooth module to the iPad or Android smartphone.

The nodes are powered by 400 mAh rechargeable battery. The BLE112 is a Bluetooth Smart module targeted for low-power sensors and accessories. It integrates all features required for a Bluetooth Smart application, including Bluetooth radio, software stack, and GATT-based profiles. The frequency range it uses is 2.402 GHz to 2.480 GHz and the maximum simultaneous Bluetooth LE (low energy) connection is 8. Data from the sensors are then transmitted via Bluetooth to the iPad.

Although we are currently using a compound sensor, i.e. a combination of range-of-motion sensors that comprises of accelerometer, gyroscope and magnetometers that may be sourced from the market, it can be envisaged that with the right volume and market size for our product and system, these core set of different sensors may be designed to be integrated into a single integrated circuit or microchip and fabricated as a much compact device as a matter of semiconductor engineering.

Compensation

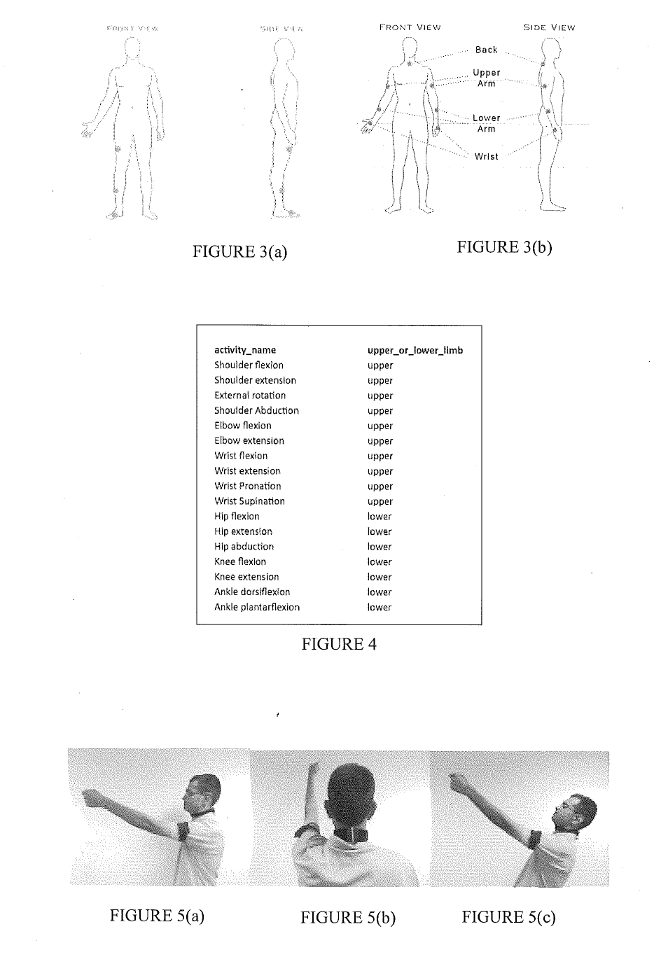

FIG. 3 (comprising FIG. 3(a) showing sensor placement on lower limb, and FIG. 3(b) showing sensor placement on upper limb). Sensor position at “Back” location indicates the sensor node placed at the back of the neck, i.e. nape, which may be used to detect compensation. In order for the system to detect compensation, a sensor node is worn at the “Back”, i.e. nape position. During exercise, the initial position of the patient (both limb node and nape sensor node) is captured and stored as the baseline. Once exercise begins, the limb sensors will capture any deviation from the initial position and measure the angle of deviation. For the neck sensor, any movement from the baseline is considered as compensation.

This compensation detection by a single strategically placed sensor is one of the key features of our tele-rehabilitation system. With this sensor node placement, we are able to tell if a patient doing the rehabilitation exercise has done it correctly without using other parts of his body for compensation. By strategically placing the compensation sensor node on the patient's nape as representative of the part of the patient's trunk, the system is able to detect if the patient is making motor compensation, such as with the use of his trunk, to compensate for his movement in yaw, pitch and roll directions while performing the prescribed exercises.

By using sensors that able to transmit motion sensed wirelessly to be processed, and strategically placing the compensation node sensor to monitor most types of motor compensation, two sensors are considered sufficient and the user does not have to don multiple sensors tethered with wires which would encumber movement and be distracting. With our method, the strategically placed compensation node would be able to provide crucial compensation information to the therapist as well as the user on whether the exercise is being performed correctly.

Preferably, inertia measurement unit (IMU) devices are used to allow the system to track upper- and lower-limb exercises in the prone, sitting, and standing positions. The use of IMUs allows the system to track exercises that require the patient to be in different starting positions (i.e. lying down on a bed, sitting in a chair, [or] standing. This is a significant advantage compared to systems based on the Microsoft Kinect, which have problems tracking exercises when the patient is lying down, and when the patient is sitting. The Kinect systems are also unable to identify the limbs of the patient correctly when there is a caregiver standing close-by to assist the patient.

For the user, the compensation information may be provided in form of biofeedback which might be any one or combination of visual cue (such as flashing light or screen), audio (beep or alarm) or vibration depending on which sensory perception is still functioning for that patient as well as the location of the compensation node or tablet's display and audio capabilities. For the therapist, the data on instantaneous movement as well as collation of data on a series of repetitive movement may provide a detailed overview of each exercise and how the patients are compensating or progressing.

Prototype and Trials

The devices have been tested on stroke patients (e.g. real-time monitoring of upper and lower limb movements). The exact type of task and the sensors required to measure, quantify a patient's improvement and monitor the various exercises are designed by the therapists, doctors, with patients' feedback. For the present patent disclosure, a list of exercises for upper and lower limbs is shown in FIG. 4.

For a randomized control trial (RCT) the initial prototype system has been tested on 15 patients from Ang Mo Kio-Thye Hua Kwan Hospital (AMKH), Bright Vision Hospital (BVH) and Singapore General Hospital (SGH). One of the immediate benefits is that patients are more motivated with supervised rehabilitation, despite carrying out the exercises at home. Real-time feedback on the patient's exercises is also provided to the patients.

Therapists can also review the patient's performance based on metrics generated from the data, whether they are doing the exercises correctly. In addition, video recordings of patients doing the exercises will also be provided. Based on these information, the therapist can adapt the exercises accordingly as the patients progresses in the rehabilitation period. The system can be easily configured for other type of rehabilitation such as wrist, knee, or hip rehabilitation.

Tele-rehabilitation is also achieved via real-time video conferencing in addition to the physical and tactile data from the sensors being transmitted to the clinicians via the Internet. The real-time feedback information from the wearable wireless sensors allows patients to monitor their own progress. Our system is also flexible enough to accommodate variations in the exact type of tasks or exercises to be prescribed to different users depending on each one's disability and need for rehabilitation which may be further designed or customized by doctors, therapists and engineers with the patients' feedback.

As the system is currently being tested in a randomized control trial (RCT), so it will be one of a very small number of rehabilitation systems that has rigorous clinical data to demonstrate its efficacy. In addition, because the system was developed very closely with doctors and therapists, we have received very positive feedback regarding our feature set from other therapists who have used the system, as well as from patients in our RCT. This gives us confidence that the system will be well received once it is commercialized.

We demonstrate the compensation detection with the following data. Consider a patient doing a shoulder flexion exercise as illustrated in FIG. 5 which illustrates a shoulder flexion exercise: FIG. 5(a) shows the side view of shoulder flexion, while FIG. 5(b) shows the backview of shoulder flexion, and FIG. 5(c) shows the shoulder flexion with front-back compensation. In FIGS. 5(a) and 5(b) is illustrated the side and back view of the patient raising his arm when doing shoulder flexion. The key information that the system captures is the maximum angle between the arm and the vertical axis. FIG. 5(c) shows the patient raising his arm while leaning backwards—he is compensating for his arm movement by moving his trunk backwards and hence appears to achieve a larger shoulder flexion angle.

This undesirable compensation is clearly shown in the graphical plot in FIG. 6 where it is shown that there is front-back compensation during the 6^{th} to 10^{th} repetitions, in particular, the patient leaning back is shown in grey bar below the x-axis of the front-back compensation plots. In the plot of shoulder flexion, the first row shows the angle achieved during shoulder flexion. The second, third and fourth rows shows front-back, left-right and rotational compensation. It is clear that on day three, there is front-back compensation during the 6^{th} to 10^{th} repetition and left-right compensation in the last four repetitions.

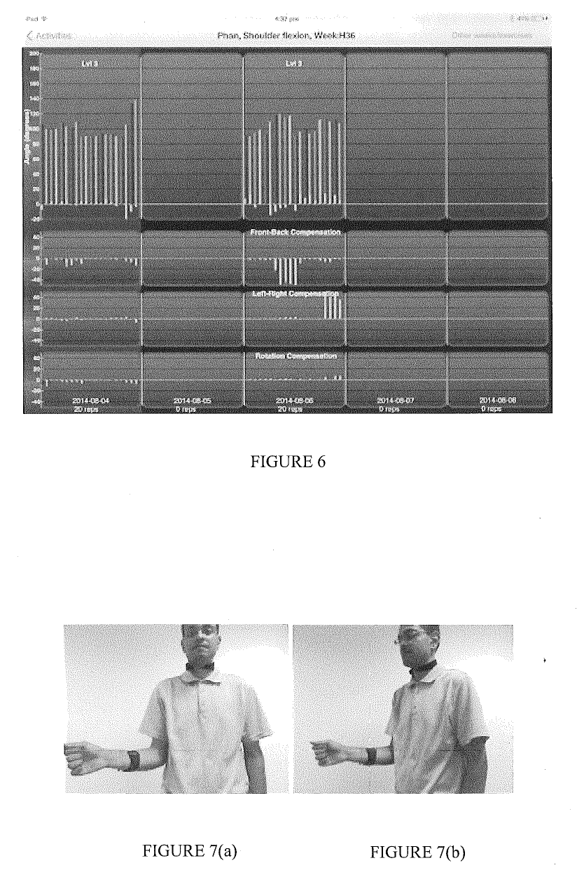

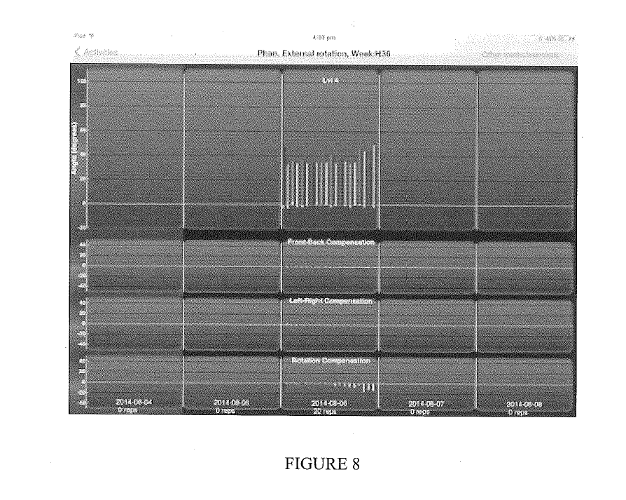

In FIG. 7, where external elbow rotational exercise is prescribed, FIG. 7(a) illustrates the front view of external rotation without rotational compensation of the trunk, and FIG. 7(b) shows the front view of external rotation with rotational compensation of the trunk. The data captured by the iPad clearly demonstrates this, as shown in FIG. 8. In this case, the patient is rotating his trunk to his right while doing external rotation thus achieving a larger angle than without compensation.

The data plot of this external rotation, as shown in FIG. 8, is clear that on day three, there is rotation compensation during the last 10 repetition. In this case, the patient is rotating his trunk to his right while doing external rotation thus achieving a larger angle compared to rotation without compensation.

The ability to monitor and measure accurately, comfortably, and continuously the progress of a patient undergoing supervised rehabilitation at home without cumbersome wires or boxes tethered around their activities has the potential to revolutionize health therapies and services. We have developed an affordable, wireless prototype for home tele-rehabilitation therapy system that is well tolerated by stroke patients, and can be performed over the duration needed to obtain results. Therapists can assess and talk to their patients via video conferencing; in addition, video, physical and tactile data from the sensors are transmitted to the clinicians via the Internet.

With the data, the system produces metrics to quantify the progress, as well as how well the patient is doing the exercises. This allows the therapist to quickly assess how many exercises the patients performed, if the patient was performing any of the exercises incorrectly, and adapt the rehabilitation exercises accordingly to help the patient. The availability of real-time feedback from the wearable wireless sensors also allows patients to monitor their own progress. In addition, the system can also be used initially to quantify the disability of the patients. Finally, we would like to highlight that our invention is able to provide active rehabilitation for these patients that results in tangible improvement in meaningful motor functions.

1. A method for assisting a user performing musculoskeletal movement in rehabilitation exercise, the method comprising
motion-sensing a first part of user's body to be exercised;
motion-sensing a second part of user's body not intended to be moved during said first part of user's body being exercised; and
alerting said user of undesirable compensatory movements sensed of said second part of user's body in relation to the first part of the user's body being exercised.

2. A method for assisting a user performing musculoskeletal movement according to claim 1 wherein the motion-sensing of at least one of the first part and second part of user's body is transduced into a wireless signal and transmitted for data processing and data storage.

3. A method for assisting a user performing musculoskeletal movement according to claim 1. wherein the alerting of undesirable compensatory movements is made in the form of real-time biofeedback to the user, including any one or combination of visual, audio or vibration, said biofeedback of intensity sufficient to be perceivable by said user as a stroke patient.

4. A method for assisting a user performing musculoskeletal movement according to claim 3 wherein the alerting of undesirable compensatory movements to the user is made as part of the data processing.

5. A method for assisting a user performing musculoskeletal movement according to claim 1 wherein the first part of user's body is a limb to be exercised and the second part is a point on user's body most likely subject to undesirable compensatory movements.

6. A method for assisting a user performing musculoskeletal movement according to claim 5 wherein the motion-sensing of the first part of user's body is sensed at a volitional torque measurable point of the limb to be exercised, and the motion-sensing of the second part of the user's body against undesirable compensatory movements is sensed at the user's neck.

7. A method for assisting a user performing musculoskeletal movement according to claim 2 wherein the data processing includes any one or combination of
alerting of undesirable compensatory movements by the user;
storing and plotting the data for visual representation of the rehabilitation exercise;
transmitting aforesaid data to a rehabilitation supervision centre; and optionally
receiving data from said rehabilitation supervision centre.

8. A method for assisting a user performing musculoskeletal movement according to claim 6 comprising
detecting said undesirable compensatory movements at the user's neck in relation to the limb's movements,
transducing both movements into signals and transmitting said signals to be processed into visual representation of the rehabilitation exercise session, wherein
said visual representation comprises plotting each of the movements in series and align both movement series by time-frame.

9. A method for assisting a user performing musculoskeletal movement according to claim 7 wherein the data processing includes video conferencing between the user performing the rehabilitation exercise and the rehabilitation supervision centre enabling a therapist to view said user's exercises and providing instructions or guidance to said user in real-time.

10. A method for assisting a user performing musculoskeletal movement according to claim 9 wherein data transmission is conducted via web-based server processing accessible by said user and therapist.

11. A method for assisting a user performing musculoskeletal movement according to claim 10 wherein one therapist is in multiple simultaneous data transceiving sessions with a plurality of such connected users.

12. An apparatus for assisting a user to perform musculoskeletal movement in rehabilitation exercise, the apparatus comprising
a first motion sensor attachable to a first part of user's body to be exercised;
a second motion sensor attachable to a second part of user's body not intended to be moved during said first part of user's body being exercised; and
an alarm alerting said user of undesirable compensatory movements sensed of said second part of user's body in relation to the first part of the user's body being exercised.

13. An apparatus for assisting a user to perform musculoskeletal movement according to claim 12 wherein at least one of the first and second motion sensors transduces motion sensed into wireless signal and transmitting it to a computing device for data processing and storage.

14. An apparatus for assisting a user to perform musculoskeletal movement according to claim 13 wherein the alarm alerts the user of making undesirable compensatory movement by way of real-time biofeedback to said user, including any one or combination of visual, audio or vibration, said biofeedback of intensity sufficient to be perceivable by said user as a stroke patient.

15. An apparatus for assisting a user to perform musculoskeletal movement according to claim 14 wherein the alarm is triggered as a result of the computing device having processed the data received from the motion sensor indicating undesirable compensatory movements being sensed.

16. An apparatus for assisting a user to perform musculoskeletal movement according to claim 12 wherein the first part of the user's body attachable with the first motion sensor is a limb to be exercised and the second part of the user's body attachable with the second motion sensor is a point on the user's body that is most likely to be subject to undesirable compensatory movements.

17. An apparatus for assisting a user to perform musculoskeletal movement according to claim 16 wherein the motion sensor attachable on the first part of user's body is attached to a volitional torque-measurable point of the limb to be exercised, and the motion sensor attachable to the second part of the user's body for detecting undesirable compensatory movements is attached to the user's neck.

18. An apparatus for assisting a user to perform musculoskeletal movement according to claim 13 wherein the computing device provides data processing for any one or combination of
alarm triggered upon undesirable compensatory movements by the user;
data storage and plotting to generate visual representation of the rehabilitation exercise;
transmitting aforesaid data to a rehabilitation supervision centre; and optionally
receiving data from said rehabilitation supervision centre.

19. An apparatus for assisting a user to perform musculoskeletal movement according to claim 17 wherein
the motion sensor detecting said undesirable compensatory movements is adapted to be donned on the user's neck, said detection is in relation to the limb's movements,
the computing device receiving signals of both movements is operable to process said signals into visual representation of the rehabilitation exercise session, wherein
said visual representation comprises charts of each of the movements in series and both movement series' charts are aligned by time-frame.

20. An apparatus for assisting a user to perform musculoskeletal movement according to claim 19 wherein the computing device includes a video camera enabling video conferencing between the user performing the rehabilitation exercise and the rehabilitation supervision centre, thus allowing a therapist to view said user's exercises and providing instructions or guidance to said user in real-time.

21. An apparatus for assisting a user to perform musculoskeletal movement according to. claim 20 wherein data transmission between the computing device at user's end and the rehabilitation supervision centre is conducted via a web-based server which is accessible by said user and therapist.

22. A plurality of apparatuses each for assisting a user to perform musculoskeletal movement according to claim 21 in simultaneous data transceiving sessions of multiple users with a therapist at the rehabilitation supervision centre.
